(19) United States
(12) Patent Application Publication
(10) Pub. No.: US 2010/0234742 A1
(43) Pub. Date: Sep. 16, 2010

(54) System and Method for Blood Pressure Management

(75) Inventor: Kin-Yuan Lin, Taipei (TW)

Correspondence Address: SHOEMAKER AND MATTARE, LTD, 10 POST OFFICE ROAD - SUITE 100, SILVER SPRING, MD, 20910, US

(73) Assignee: MICROLIFE INTELLECTUAL PROPERTY GMBH, Widnau (CH)

(21) Appl. No.: 12/161,377

(22) PCT Filed: Nov. 2, 2006
(86) PCT No.: PCT/EP2006/068039

§ 371 Date: Jul. 18, 2008

(30) Foreign Application Priority Data
Jan. 20, 2006 (EP) 06100651.6

(51) Int. Cl. A61B 5/022

(52) U.S. Cl. 600/490

(57) ABSTRACT

A system (1) for blood pressure measurement at the point of care comprises a blood pressure measuring device (10) for measuring the blood pressure of an individual (I) and seat (20) for properly positioning the individual (I). The seat (20) includes means (21) for ensuring correct position of the individual's feet and legs. The blood pressure measuring device (10) is arranged at a location (12) physically separated from a data collecting device (30). The blood pressure measuring device (10) is provided with a communication interface (11) for transmitting data to the collecting device (30).

The invention relates to a system and a method for blood pressure measurement. Hypertension is one of the major health problems in modern societies. Reliable determination of blood pressure of individuals is one of the most important conditions for appropriate treatment of hypertension. Inappropriate treatment or medication may be a result from inaccurate measurements. It has been suggested by several authors that home measurements may be most suitable for accurate blood pressure monitoring (see e.g. Laurie Barclay, “Home measurement best for accurate BP monitoring”, BMJ. 2002; 325:254-257 or Judy Possidente Kaufman et al., “The role of home blood pressure monitoring in hypertension control”, J Clin Hypertens 3(3):171-173).

It is generally known that the so called white coat effect leads to inaccurate blood pressure measurements done by physicians. Too high blood pressure readings done by general patricians should not be used to make decisions about treatments. However, according to Kaufman, there are still some problems with self reported blood pressure measurements. One major problem is erroneous reporting. Consequently, prudent clinicians will augment home reading data with periodic clinic measurements.

According to Pickering (Pickering et al., “Recommendations for blood pressure measurement in human and experimental animals”, Hypertension, January 2005, page 144,) surveys have shown that physicians and other health care providers rarely follow established guidelines for blood pressure measurements. There are several reasons why usual clinic readings may give poor results. This may not be only due to poor technique but also because they typically only consist of one or two individual measurement. However, beat to beat blood pressure variability is such that a small number of readings can only give a crude estimate of the average level. A plurality of measurements therefore will be needed in order to have reliable results.

Furthermore, measuring errors may be caused by wrong posture. In particular, back support may affect seated blood pressure determinations (see Cushman et al., “Effect of back support and stethoscope head on seated blood pressure determinations”, AM J. Hypertens. 1990; 3:240-241). Also crossing the legs may raise systolic pressure (Peters et al., Blood Press Monit. 1999; 4:97-101, “The effect of crossing legs on blood pressure: randomised single-blind cross over study”). Further authors (Terrent et al., “Epidemiological perspective of body position and arm level in blood pressure measurement”, Blood Press. 1994; 3:156-163) have concluded that deviations from the recommended body position and arm level may be clinically relevant. It can be followed that reliable blood pressure measurements can only be achieved if a plurality of measurements are done with a correct posture. Furthermore, the effect of white coat hypertension needs to be considered. In the recommendations for blood pressure measurement in humans and experimental animals (Pickering, Hypertension, January 2005, page 142-161) recommendations for correct blood pressure measurements are given. In particular, the individual should be comfortably seated with legs uncrossed and back and arms supported. The patient should be seated in a chair. At an initial visit, blood pressure should be measured in both arms. Furthermore, the individual must relax before initial measurements. Furthermore, a plurality of readings is suggested in order to form more reliable averages. Reduction of white coat hypertension effect can be achieved by automatic determination of blood pressure with automatic blood pressure measurement devices.

A further problem in view of achieving reliable blood pressure measurement results is based on the fact that depending on the measurement site, different measurement results may be found. In particular, inter-arm differences between the left and right arm may exist. According to Deirdre Lane (Deirdre Lane et al, “inter arm differences in blood pressure: when are they clinically significant?”, J Hypertens 20;1089-1095, 2002), O'Brien (O'Brien et al, “European Society of Hypertension Recommendations for conventional, ambulatory and home blood pressure measurement”, J Hypertens 2003, Vol 21 No. 5, page 823) or Gosse (P. Gosse, “Blood pressure should be measured in both arms under first consultation”, J Hypertens 2002; 20:1045-1046), inter-arm differences in blood pressure measurements may be clinically significant and thus blood pressure measurements should be made in both arms during a first consultation. Main purpose of these suggestions is prevention of misdiagnosis of hypertension due to normal differences in the blood pressure between the arms. Furthermore, too high differences may be an indication for existence of arterial diseases.

While there are many recommendations and suggestions for blood pressure measurement, currently there is no system available for carrying out measurements in accordance with such recommendations.

It is therefore an object of the present invention to overcome the drawbacks of the prior art, in particular to provide a system and a method for blood pressure measurements which allow to make accurate and reliable blood pressure measurements in a doctor's office. In accordance with the present invention, these and other objects are solved with a system and a method according to the independent patent claims.

According to a first aspect of the invention, there is provided a system for blood pressure measurement which is particularly suitable for measurement at a point of care. While it is generally accepted that home blood pressure monitoring may be a preferred choice, practitioners may wish to verify home measurements with there own measurement. Therefore, the present system proposes a device for point of care measurement, e.g. for measurement at a doctor's office or at a pharmacy. The system comprises a blood pressure measuring device for measuring the blood pressure of an individual. Furthermore, the system is provided with a seat for the individual adapted for properly seating the individual in view of correct blood pressure measurement. The seat includes means for ensuring a correct position of the individual's feet. This typically maybe a foot rest or also a foot pad including feet printing in order to indicate appropriate position. The system according to the present invention makes sure that measurements of blood pressure can only be done when the individual is properly positioned. By integrating or combining a blood pressure measuring device and an appropriate seat, correct position is automatically achieved.

According to a further aspect of the invention, there is provided a blood pressure measurement system which comprises a blood pressure measuring device for measuring the blood pressure of an individual. The blood pressure measuring device has a communication interface for transmission of measured blood pressure data. The system further comprises a data collection device which has a communication interface for receiving data transmitted by the blood pressure measuring device. According to the present invention, the data collecting device is arranged at the point of care but at a location physically separated from a location of the blood pressure measuring device. Physical separation is made in such a way that a direct contact between a doctor or a care person and the individual during blood pressure measurements can be prevented. By arrangement of the blood pressure measuring device at the point of care but physically separate from a care person, the white coat hypertension effect can be reduced or avoided. In particular, the blood pressure measuring device can be arranged at a separated location such as a room or corner which is particularly adapted for blood pressure measurements according to predetermined measurement criteria. In particular, there can be provided a seat as mentioned above for ensuring proper posture of the individual. Furthermore, the separated location can be designed in order to allow the individual to relax. In particular, the location may be a quiet room visually and/or acoustically isolated from the location of the data collection device or from a doctor's office.

According to another aspect of the invention there is provided a system for blood pressure measurement which basically consists of a blood pressure measuring unit. The system is provided with a first cuff for blood pressure measurement, adapted to be placed around a patient's left art and connected to or connectable to the unit. The system is also provided with a second cuff for blood pressure measurement adapted to be placed around a patient's right arm and connected to or connectable to the unit. According to this aspect of the invention, the blood pressure measuring unit is provided with means for parallel measurement of the blood pressure with said first and said second cuff. While such a system is particularly preferred in accordance with a system as described hereinabove, obviously, a system having two cuffs for parallel measurement maybe advantageous also in other context. In particular, such a system helps to make parallel measurements during a first visit of the patient at the point of care and thus helps to determine significant inter-arm differences.

The device according to this aspect can be used to prevent misdiagnosis of hypertension. It also ensures that the effectiveness of the therapy will be accurately monitored by measuring the BP in the same arm on all subsequent occasions.

According to a preferred embodiment of the invention, this system may be operatable in two operating modes. One of these operating modes is a so called initial visit mode. In this initial visit mode, an initial visit loop is carried out. In this initial visit loop, a parallel measurement of the blood pressure is made with the first and the second cuff. Other modes in addition to the initial visit mode may allow standard uses of such a device.

A decision if a measurement is an initial measurement can be made by entering appropriate information into the system. The system may be provided with a specific input interface for this purpose. In particular, measurements of blood pressures on the right and on the left arm and formations of differences between these measurement values will be done in the initial visit loop.

According to another preferred embodiment of the invention, in addition to the initial visit mode, the system may be operatable in at least one of a manual or auscultation mode and a follow up mode. In the manual (or auscultation) mode, current pressure values in at least one of the cuffs are instantly displayed on a display of the unit. Thereby, by use of a stethoscope, a doctor may make manual blood pressure measurements. These measurement values may be used for verification of the automatic measurements or may also be entered into the system in accordance with a specific preferred embodiment.

In the follow up mode, the system is working in a traditional automatic blood pressure measurement mode. Measurements are determined on the basis of pressure signals provided by one of the first and the second cuff in a known automatic method, in particular according to the oscillometric method.

In this context, the system may be preferably provided with a switching means for switching the system between the above mentioned operating modes.

Furthermore, the system may comprise means for automatically determining a preferred measurement arm for subsequent blood pressure measurements in the follow up mode. Preferably, the system may be provided with a display means for indication of the preferred measurement arm. Typically, determination of a preferred arm may be based on the higher reading between the two arms. If a reproducible difference is evident, the arm with the highest BP recording should be used in future for all BP measurements.

According to a further preferred embodiment of the invention, the system comprises means for forming averages of measurement values of a plurality of subsequent measurements. This allows to further increase reliability of the results.

The system further may be provided with means for determining the difference of blood pressure values measured on the right arm and on the left arm of the individual.

The system further may be designed with means for making a pause of a predetermined or predeterminable length between subsequent measurements. By making a pause, it is made sure that the individual is brought to a relaxed rest condition before a further measurement is made.

According to a further preferred embodiment of the invention, the system may be provided with an input interface for entering blood pressure data during this initial visit loop. It may be preferred by some practitioners to also consider manually made blood pressure measurements, e.g. blood pressure measurements based on auscultatory measurement, preferably in the auscultation mode. These measurements will be stored in the system and will be associated to a specific individual.

According to a further preferred embodiment of the invention, the seat of the system according to the invention includes also at least one of an arm or a back support for the individual. This further enhances proper posture of the individual.

It is further preferred for the data collection device to include a communication interface for transmitting collected blood pressure data to a central data base or a central storage means. Such an additional communication interface allows to store data of the individual in a centralised manner such that a plurality of care persons such as doctors, nurses, pharmacists or also family members can access or update the individual's data.

According to still a further preferred embodiment of the invention, the system according to the invention may include means for generating reports in accordance with predetermined reporting criteria. In particular, in view of pay for performance programs for doctors or also in view of medication therapy management programs for pharmacists, such reports might be useful. If the device is designed for automatically generating such reports, it will be much easier for the doctor or pharmacist to follow the recommendations or rules of such programs. Typically, such reports may be created as electronic files or also in paper form.

According to a further aspect of the invention, there is provided a method for blood pressure measurement of an individual at a point of care. In a first step, the individual is placed properly at a location in such a way that there is no direct contact between the individual and a doctor or a care person during blood pressure measurement. Subsequently, a blood pressure measurement for gathering blood pressure data of the individual is automatically carried out with a blood pressure measuring device in absence of a doctor or of a care person. Therewith, inaccurate measurements because of the white coat effect are avoided. The gathered blood pressure data are subsequently transmitted to a data collection device which is arranged at the point of care but at a location physically separated from the blood pressure measuring device.

According to a further preferred method of the invention, prior to the measurement of the blood pressure of the individual, one or plural instructions may be given to the individual. In particular, instructions such as to have legs and arms supported, to place feet in a predetermined manner, in particular on a predetermined foot rest or instructions to rest for a predetermined time may be given to the individual. By automatically providing such instructions, proper posture of the individual may be ensured. These instructions can be generated either automatically by the system or by a care person.

According to still a further embodiment of the invention, prior to blood pressure measuring, in a judgement step, a judgement as to whether or not this is the individual's first or initial visit is made. Depending on the answer, different measurement routines may be appropriate. In particular, an initial visit loop can be carried out if this is the individual's initial visit. In this initial visit loop, it is e.g. possible and preferred to enter manually measured blood pressure data into the system before automatic measurements will be made. It is also possible to carry out blood pressure measurements of the individual on the individual's left and right arm during the initial visit loop in order to form respective blood pressure values. While such a measurement on both arms is preferred in a method as described hereinabove, obviously, such a method can all be advantageous in context with other methods. Preferably, measurements on both arms are made simultaneously. Preferably, it is also possible to form a difference between the measurements on the left and on the right arm.

If differences between the 2 arms are found, blood pressure should be checked in both arms at the first examination. This may be helpful in detecting coarctation of the aorta and upper extremity arterial obstruction. When there is a consistent interarm difference, the arm with the higher pressure will be used.

According to a further preferred embodiment of the invention, at least two subsequent measurements of blood pressure values are made. An average of measurements of subsequent readings of the blood pressure values then may be formed.

According to still a further embodiment of the invention, a pause of a predetermined or a predeterminable length is made between subsequent measurements. It is also possible to make a pause before the initial measurement in order to make sure that the individual is at rest.

When a plurality of subsequent measurements is made, it is also possible and preferred to form a difference between subsequent measurement results. If the blood pressure values of two subsequent measurements differ too much, i.e. if a difference between blood pressure values of two subsequent measurements is above a predetermined or a predeterminable value, a further measurement may be carried out in order to exclude potentially erroneous results.

According to a further aspect of the invention, the cuffs adapted to be placed around the patient's right and left arm are each provided with an inflation valve which allows independent adjustment of the deflation rate. Depending on the strength of application of the cuff around the patient's right and left arm, the air volume in the cuff necessary to achieve a preset pressure may be slightly different. As a consequence, the time necessary to reach the preset pressure is different. Adjustment of the individual deflation rate of both valves allows to have a equal total time for deflation and inflation for both of the cuffs. Inter arm measurements thus can be completed in exactly the same time.

The invention will now be described with respect to the following embodiments and accompanying drawings, which show:

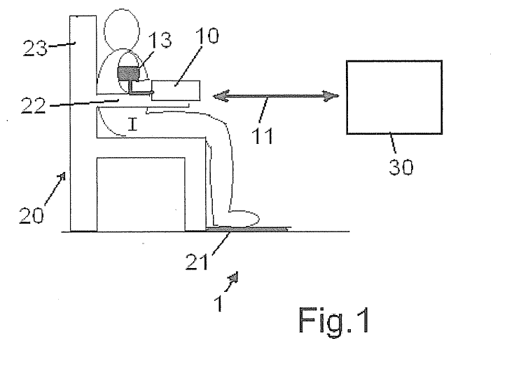

FIG. 1A schematic representation of a seat according to the present invention

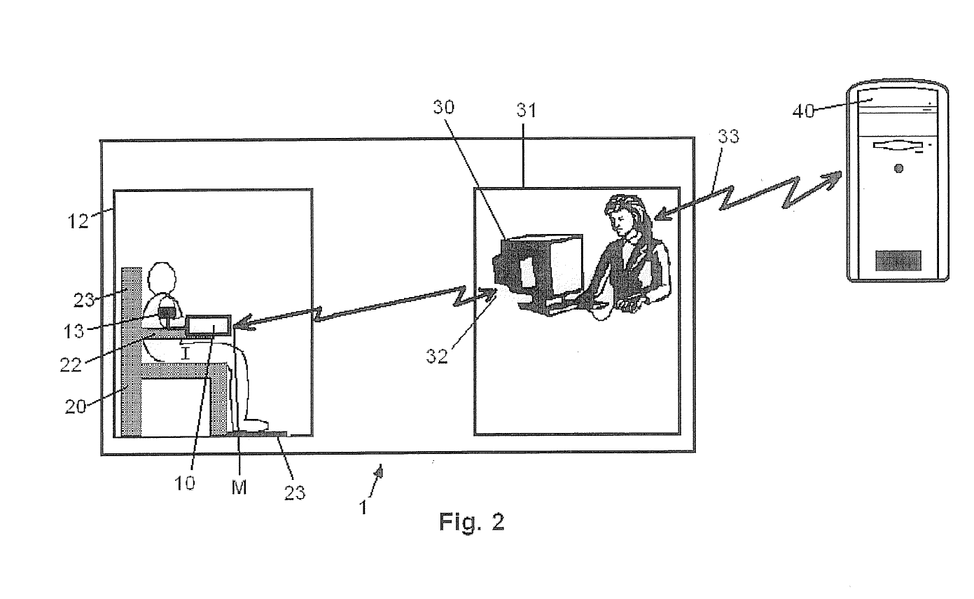

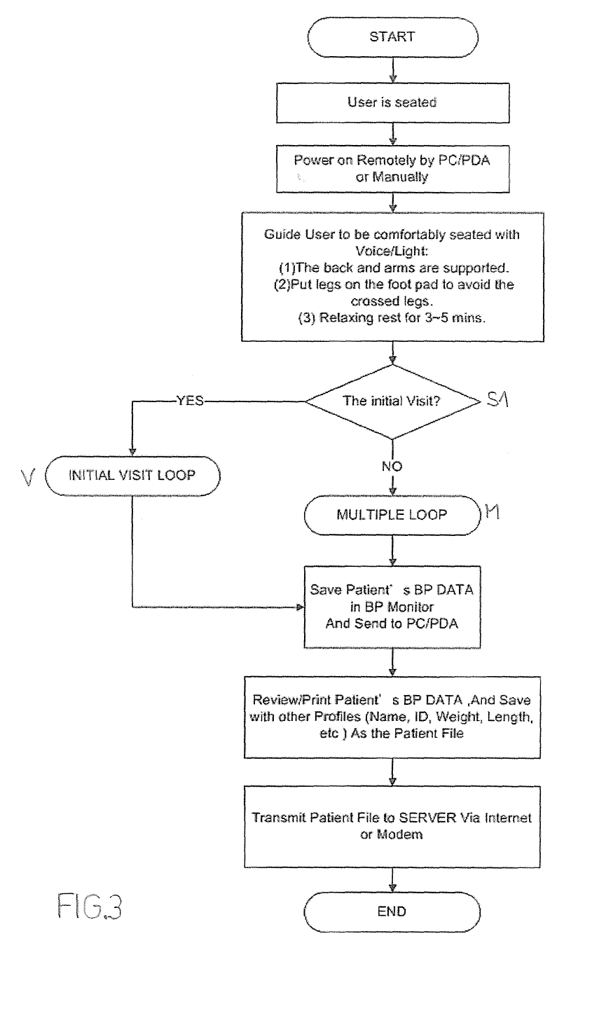

FIG. 2 A systematic representation of a system according to the present invention

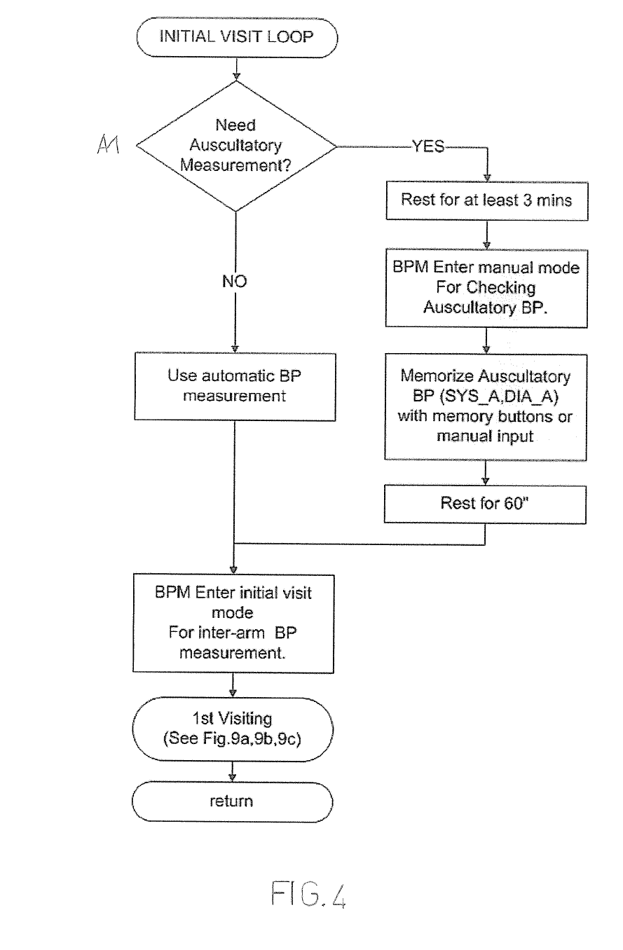

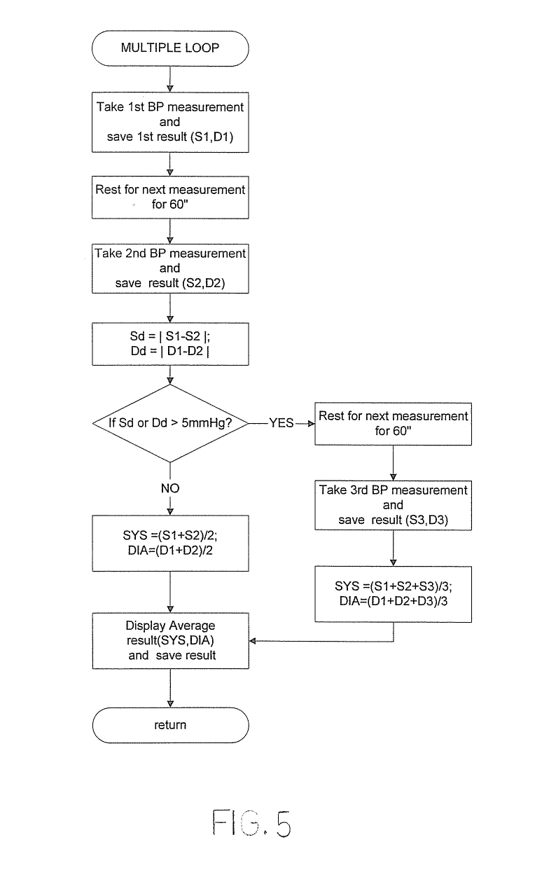

FIGS. 3-5 Flow charts of operations of a method according to the present invention

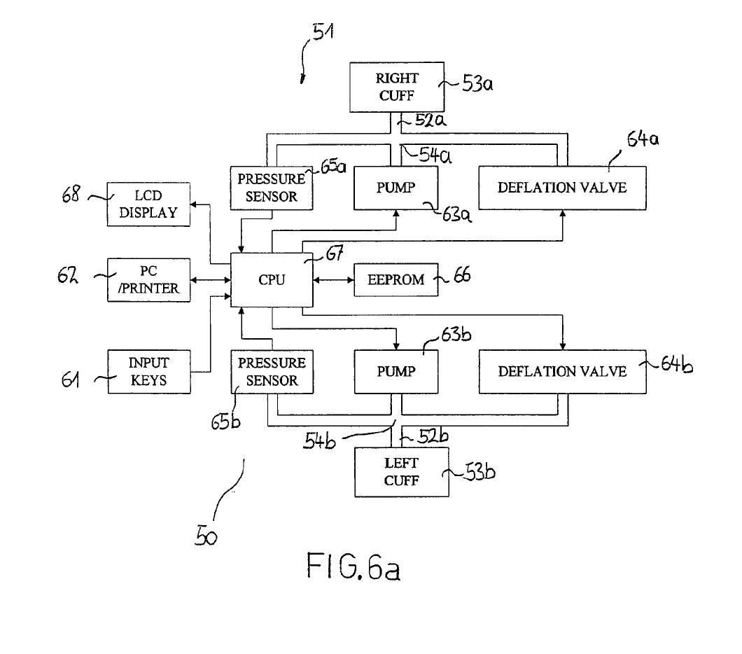

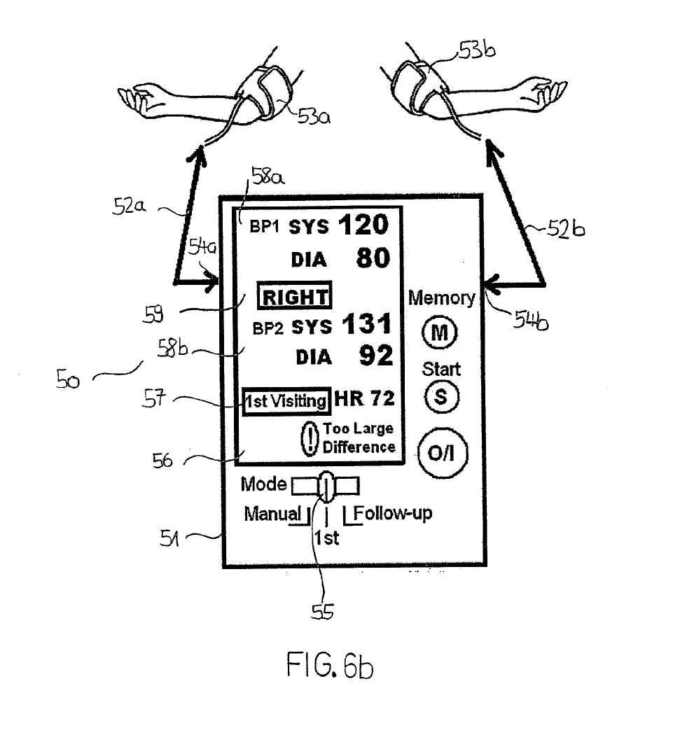

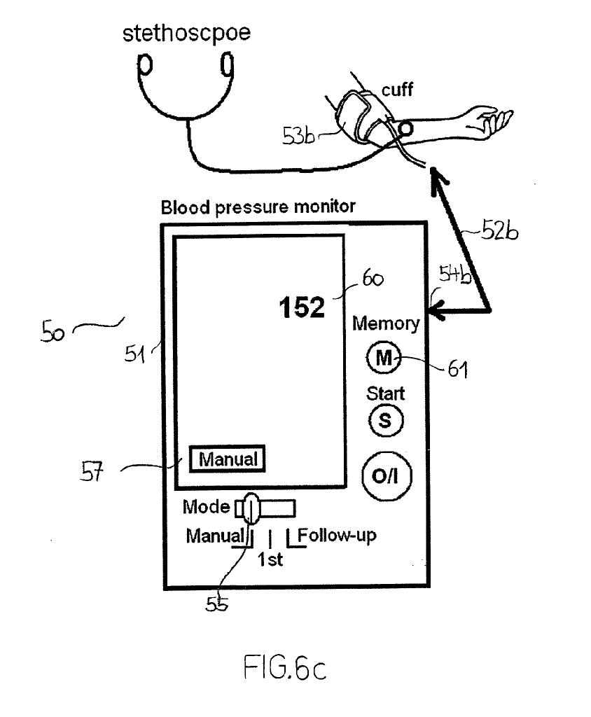

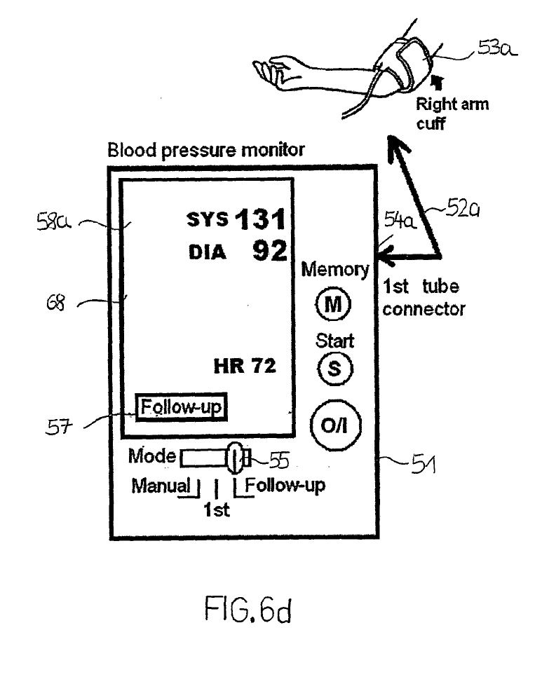

FIG. 6a A block diagram of a system according to the invention

FIG. 6b A schematic representation of an alternative embodiment of a system according to the present invention in a first visit mode

FIG. 6c A schematic representation of the system of FIG. 6a in a manual mode

FIG. 6d A schematic representation of the system of FIG. 6a in a follow up mode

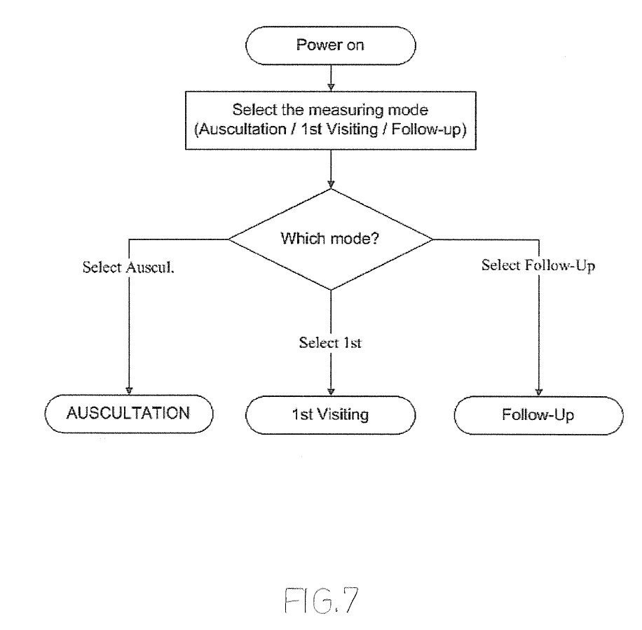

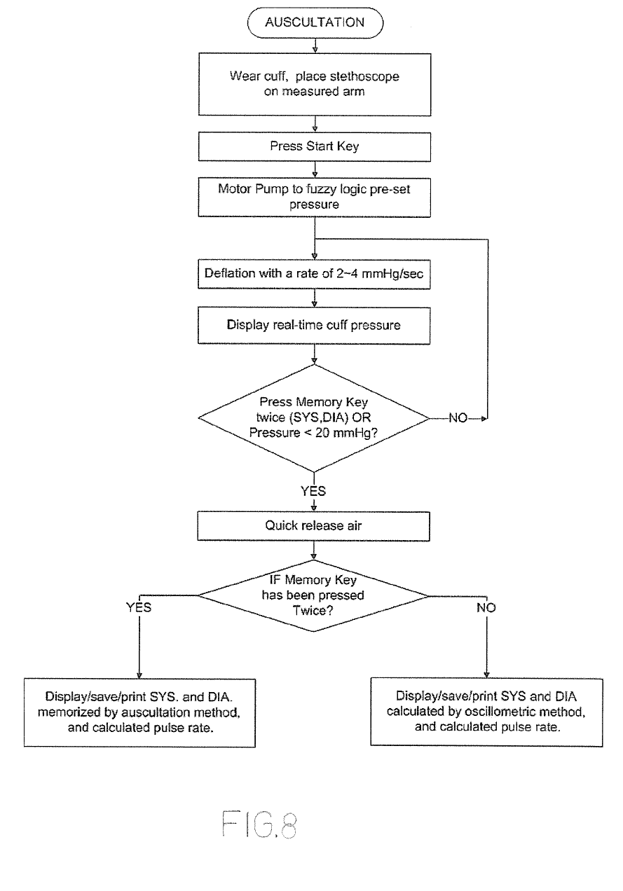

FIG. 7 A flow chart of operation of the system as shown in FIGS. 6a and 6b

FIG. 8 A flow chart of the auscultation or manual mode of a system in accordance with FIG. 6b

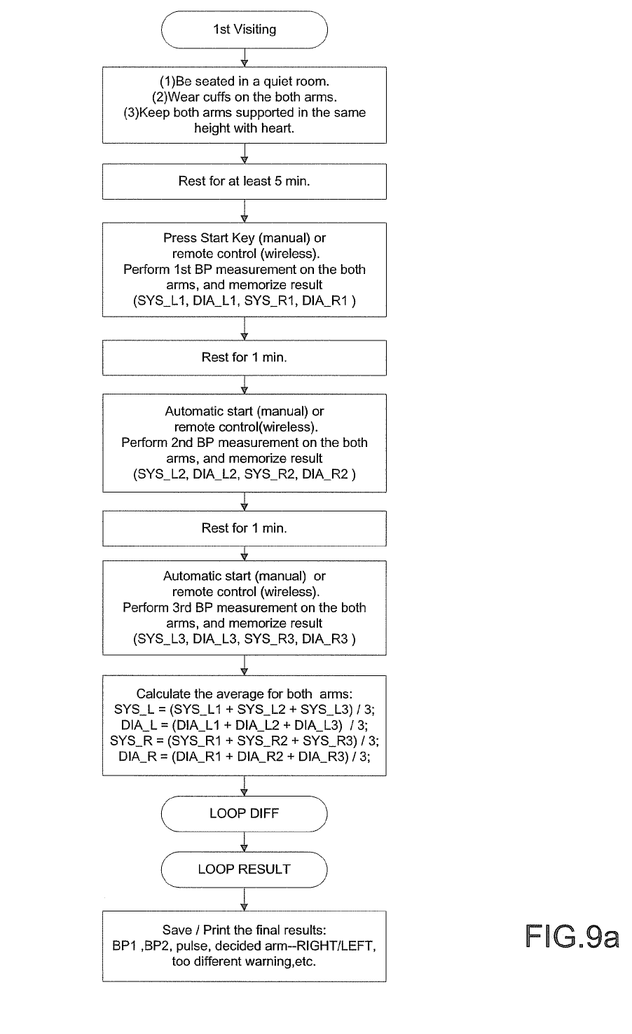

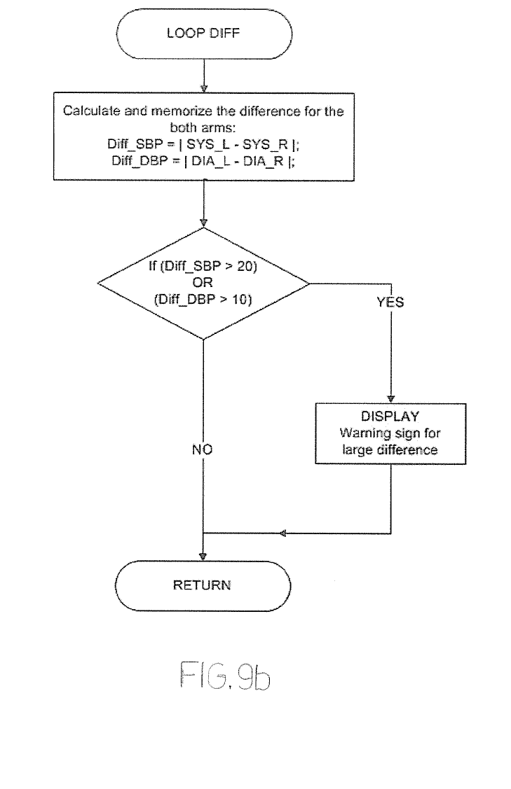

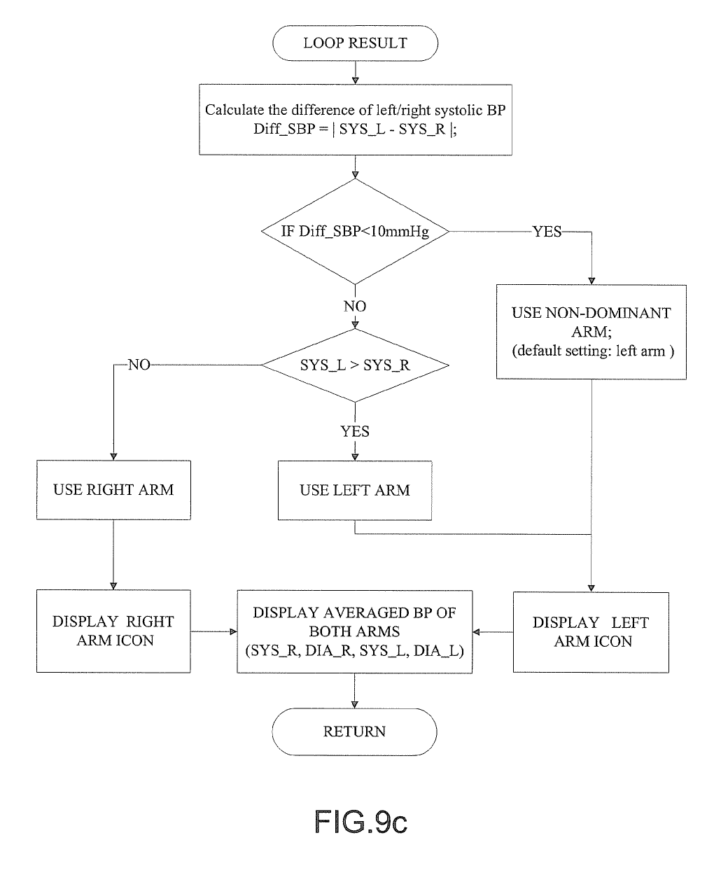

FIG. 9a A flow chart of an alternative embodiment of an initial visit loop

FIG. 9b A flow chart of a loop difference procedure as shown in FIG. 9a

FIG. 9c A flow chart of a loop result procedure as shown in FIG. 9a and

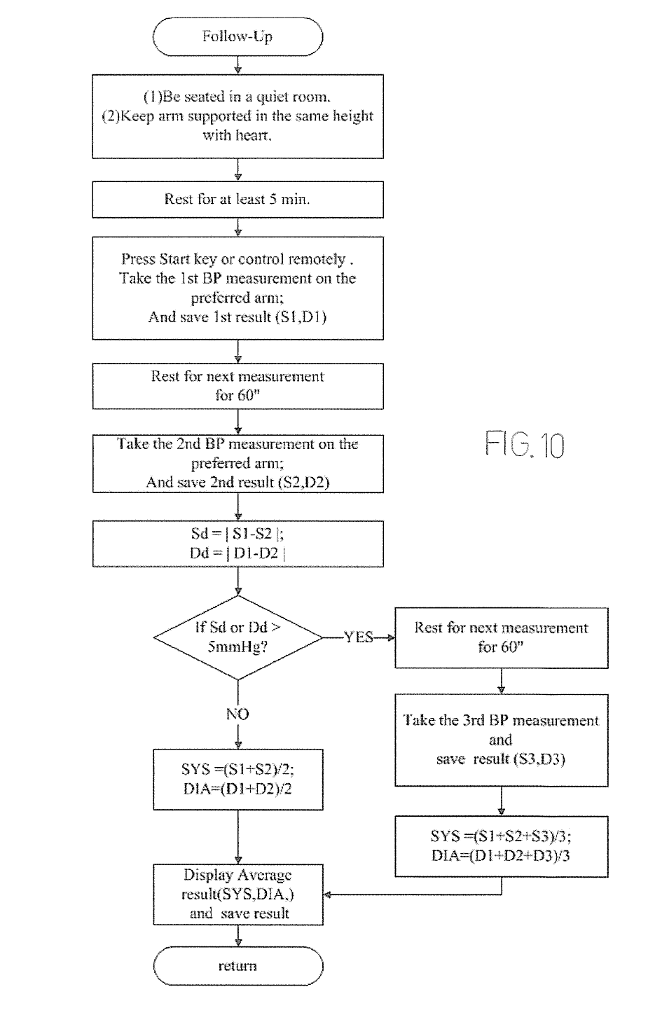

FIG. 10 A flow chart of operation of the system in accordance with FIGS. 6a and 6b in a follow up mode.

FIG. 1 schematically shows a system 1 for measuring hypertension of a patient at a point of care. The system 1 basically comprises a blood pressure measuring device 10 and a seat 20 for proper positioning of the individual I. The blood pressure measuring device 10 is designed in a substantially conventional manner and includes a cuff 13 to be placed around an upper arm of the individual I. The blood pressure measuring device 10 is connected to the cuff 13 via a tube. The blood pressure measuring device 10 includes in a conventional manner a pump, pressure sensor and microprocessor means for determining automatically the blood pressure of the patient. In particular, determination may be made in the oscillometric method.

The blood pressure measuring device 10 is provided with a data communication interface 11 for transmission of data measured with the blood pressure measuring device 10 to a data collection device 30. The data collection device 30 typically is a personal computer, a personal digital assistant or another electronic equipment. It may also be a printer for printing out the measured values. Data transmission may be through a wire connection such as USB or RS 232 or preferably wireless such as by a Blue Tooth connection.

The system 1 further comprises a seat 20 for the individual I. The seat 20 is provided with foot rests 21. The foot rests 21 include markings or indications for proper positioning of the left and the right foot. These markings (not shown) make sure that the individual I is taking a measurement with uncrossed legs in accordance with measurement recommendations. The seat 20 further includes an arm support 22 and a back support 23 for ensuring proper positioning of the individual I.

The system 1 is shown in some more detail in FIG. 2, whereas like reference numerals designate like parts. The blood pressure measuring device 1 together with the seat 20 is arranged at a quiet location 12. The data communication interface 11 allows for data transmission to the data collecting device 30 in the form of a personal computer. The data collecting device is typically arranged at a doctor's office 31 physically separated from the quiet location 12. With the system according to FIG. 2, blood pressure measurements may be taken with a patient properly seated on the seat 20 at the quiet location 12. Inaccurate measurements due to improper positioning or in view of white coat effect are avoided. A doctor seated in the doctor's office 31 may review a patient's file and measurement results. The measurement results may be stored in a data base. A further communication interface 33 such as an internet connection between the data collecting device 30 and an external data base server 40 may allow for centrally storing patient information including blood pressure measurement data.

A flow chart for carrying out the method according to a first embodiment of the invention is shown in FIG. 3. In a first step, the user or individual I is properly seated on the seat 20. The power of the system 1 is then either manually or preferably remotely turned on.

In a further step, the user is guided to be comfortably seated within the seat 20. This may be done acoustically or visually either by generating voice messages or by displaying messages on a screen. In particular, the user is instructed to have his back and arms properly supported by the back support 23 and the arm support 23. The user is further instructed to have his legs properly positioned on the foot rest 21 in order to avoid crossed legs. Furthermore, the user is instructed to relax for approximately 3 to 5 minutes. All these steps are preferably generated from a remote location or automatically in absence of a doctor or a care person.

In a subsequent judgement step S1 it is judged whether this is the individual first or initial visit. If this is the initial visit of the individual I, an initial visit loop V (see FIG. 4) is carried out. After the initial visit loop V, another rest for a predetermined period of time, approximately 3 to 5 minutes will be made. If this is not the initial visit, blood pressure measurements will be made in a multiple loop (see FIG. 5). After completion of the multiple loop M or the initial visit loop, patient blood pressure data are stored in the blood pressure measuring device 10 and sent to the data collecting device 30 through the communication interfaces 11 and 32, respectively. In a further step, the patient's blood pressure data will be reviewed by a doctor, care person or pharmacist on the data collecting device 3 and will be saved with other relevant information such as name, ID, weight, length or the like in a patient's file. In a final, optional step, this patient file may be transferred to the central server 40 through the communication interface 33.

The initial visit loop V in accordance with this first embodiment is shown in FIG. 4. In a first step A1 it is verified whether an auscultatory measurement may be necessary. If there is such a requirement, the patient is asked to rest for at least 3 minutes. Subsequently, a manual mode in the blood pressure monitoring unit 51 (see e.g. FIG. 6a, 6b) is selected. Blood pressure values are manually determined by means of a sthethoscope. These manually measured blood pressure values are entered into the system. These are typically blood pressure values achieved in traditional auscultatory methods. The manually gathered blood pressure values SYS_A, DIA_A are stored in the system by actuating memory buttons or appropriate input buttons (not shown). After entry of these data, the individual is asked to rest for about 60 seconds.

If in step A1 it is found that no auscultatory measurement is necessary, an automatic blood pressure measurement is done. Subsequently, in both cases, an initial visit loop is started for making an interarm blood pressure measurement. First visiting loop will be explained with respect to FIGS. 9a, 9b and 9c.

If this is not the individual's initial visit, a multiple loop is immediately carried out. In the multiple loop (see FIG. 5) a first blood pressure measurement is made with the measurement device 10. The measurement results S1, D1 are stored. After a rest for 60 seconds, a second measurement is made and the results S2, D2 are stored in the system. Subsequently, an absolute value of a difference Sd, Dd is formed between the measurement values of the first and the second measurement. If Sd or Dd is below 5 mmHg, an average SYS, DIA is formed of the measurement results of the first and the second measurement. These average results SYS, DIA are displayed and saved in the system. If the difference Sd, Dd is above 5 mmHg, a third blood pressure measurement will be made after another rest of 60 seconds. The measurement results S3, D3 of the third measurement will be stored and average values SYS, DIA of the three measurements will be formed and displayed.

FIG. 6a shows an alternative embodiment of a system 50 in accordance with the present invention. The system 50 for blood pressure measurement basically comprises a blood pressure measuring unit 51 with a microprocessor 67, a display 68 and a memory 66. The blood pressure measuring unit 51 is provided with a plurality of input keys 61, with a display and with two connector portions 54a, 54b for connecting tubes 52a, 52b of a first and a second cuff 53a, 53b. The first and second cuff 53a, 53b are adapted to be placed around a patient's left and right upper arm, respectively.

The device is designed for simultaneous measurement of the blood pressure of a user on the basis of pressure signals produced in the first and second cuff 53a, 53b. For this purpose, the system 50 is provided with two pumps 63a, 63b, two deflation valves 64a, 64b and two pressure sensors 65a, 65b. The function of the deflation valve is comparable to the one disclosed in WO 06/040295. The deflation rate can be adjusted by a drive voltage for performing a slower deflation rate around 2-4 mm HG/S during an auscultatory measurement. Also for simultaneous measurements on both sides, the deflation valve can use slightly different deflation rates so as to have a small difference between the left and right cuff pressure. Depending on the tightness of the cuffs applied to each arm, the air volume in the cuff may be slightly different. The time spent for pumping the cuff to a preset pressure consequently also may be different for each of the two cuffs. The use of two independent, actively controllable deflation valves allows to adjust the deflation rates during the measurement slightly in such a way that the inter arm measurement can be completed in the same time and in such a way that the total time for inflation and deflation for both cuffs is the same.

These components allow to independently measure the blood pressure of the patient on the basis of the left and on the basis of the right cuff 53a, 53b. Measurement is made in accordance with known automatic blood pressure measurement methods and will not be explained in more detail herein. Results of the measurement may be displayed on the LC display 56 as will be shown herein below or may also be transferred to a personal computer or printer for subsequent printout or display.

FIG. 6b schematically shows the system 50 in a so called first or initial visit mode.

The unit 51 is provided with a switch 55 for selection of one of three different operation modes. In the representation in FIG. 6a, the mode switch 55 is in a position selecting first or initial visit mode. In addition, the mode switch 55 may be used to select a manual mode (see FIG. 6c) or a follow up mode.

In the first visit mode as shown in FIG. 6b, measurement of the blood pressure is made simultaneously in parallel in the first and second cuff 53a, 53b.

In order to allow for reproducible measurements and also for reducing the so called “white code effect”, the unit 51 measures blood pressure three times successively (see also FIG. 9a) when operating in the initial visit mode.

At the end of the measurement, average blood pressure values determined on the basis of the left cuff 53b and the right cuff 53a are individually displayed in respective display areas 58a, 58b.

The blood pressure measurements unit 51 further comprises calculating means in the form of suitable programming of the microprocessor 67. These means are capable of forming a difference between the measurement results achieved by measurement at the right and the left arm. If this difference is too high, a warning such as “too large difference” is displayed in a difference warning display field 56. Furthermore, the arm on which the higher measurement result has been achieved is indicated in a preferred measurement arm display 59.

In the display as shown in FIG. 6b, the measurement made on the right arm with the cuff 53a has the higher value. Consequently, “right” is displayed in the preferred measurement arm display 59. This is an indication that subsequent measurements in a follow up mode should be made on the right arm.

FIG. 6c shows the blood pressure measurement unit 51 described in FIG. 6a, but in a manual measurement mode. Only one cuff 53b is connected to a connector 54b by means of a tube 52b. In the manual mode, manual determination of blood pressure is made. Automatic determination in parallel may be optionally possible. Once a measurement is started, the cuff is automatically inflated by operation of a pump (not shown) in the blood pressure unit. Subsequently, the pressure in the cuff 53b is continuously released by means of a valve (not shown). The actual pressure value in the cuff is displayed in an instant pressure display 60. By using a stethoscope, a doctor may therefore determine the blood pressure in conventional, auscultatory methods. Optionally, the deflation rate may be selected lower in the manual mode than in the automatic modes (i.e. in the first visit mode and follow up modes). A memory button 61 can be used to store two instantaneous pressure values.

When the memory button 61 is pressed for the first time, the current cuff pressure is stored as the systolic blood pressure. Upon second application of the memory button 61, the current cuff pressure is stored as the diastolic blood pressure.

FIG. 6d shows the unit 51 in a follow up mode. The mode switch 55 is set to the follow up position. In the mode display 57, the follow up mode is indicated. Blood pressure measurements are made on the basis of measurements on the right arm cuff 53a. After the end of the measurement, systolic and diastolic blood pressures and the heart rate are displayed on the display 68 in a display area 58a which is used for display of right arm measurements.

FIG. 7 shows a flow chart of the general operation of the unit shown in FIGS. 6a to 6d. When the unit 51 is turned on by application of an on/off button, it is first checked in which mode the mode switch 55 is set. If the mode switch 55 is set to the manual mode, an auscultation loop is carried out. If the mode switch 55 is set to the first visit mode, a first visiting loop is carried out. If the mode switch 55 is set to the follow up position, a follow up loop is carried out.

FIG. 8 shows a flow chart of the operation carried out by the device when the mode switch is in the auscultation mode.

In a first step, the cuff is applied to the upper arm and a stethoscope is placed on the arm where the cuff is placed.

Once the cuff and stethoscope are placed, a start key is pressed. The motor of the unit 51 then actuates a pump in order to pump the cuff 53b to a preset pressure. The preset pressure is determined by a fuzzy logic system known to those skilled in the art which will not be explained in more detail herein.

Once the preset pressure has been achieved, the pressure is decreased in the cuff 53b with a deflation rate in the range of 2 to 4 mmHg/sec. At the same time, the real time cuff pressure is displayed in the instant pressure display area 60 (see FIG. 6c).

By pressing the memory key 61 (see FIG. 6c) the currently displayed pressure values in display 60 are stored in a memory (not shown). The user should first press the memory key upon occurrence of the systolic blood pressure and for a second time upon occurrence of the diastolic blood pressure. As long as the memory key has not been pressed, deflation is continued. If the memory key has been pressed twice or if the pressure in the cuff has been reduced to below 20 mmHg and no memory key has been pressed, the pressure in the cuff is released quickly.

If the memory key has been pressed twice, i.e. if values corresponding to the systolic and diastolic blood pressure have been stored in a memory, the respective systolic and diastolic blood pressure values are displayed, saved and printed. A calculated pulse rate is also displayed.

If no memory key has been pressed a measurement in the auscultation mode is not possible. Optionally, systolic and diastolic blood pressure values determined by the oscillometric method and a calculated pulse rate may be displayed, saved or printed on a computer or printer 62. However, this step of determination of the values in the oscillometric method is only optional. If the memory has not been pressed twice, it is also possible to terminate the procedure.

FIG. 9a shows a flow chart of operation of the unit 51 when the unit 51 is operating in the first visiting mode. The operation shown in FIG. 9a is comparable to the initial visit loop as shown in the flow chart in FIG. 4. There are, however, some differences.

In accordance with the method in FIG. 9a, both measurements are made simultaneously on the left and on the right arm. Consequently, in a first step it must be verified that both arms are correctly supported, in particular supported in the same height as the heart. The patient is then asked to rest for at least 5 minutes after having been seated in the quiet room.

Once measurement is started by pressing a start key (or also by remotely controlling operation of the unit 51), a first blood pressure measurement is carried out on both arms and the respective systolic and diastolic blood pressure values are stored. After a rest for one minute, the measurement procedure is repeated twice so as to form a series of three measurement results of the systolic and the diastolic blood pressure on the basis of both cuffs.

In a next step, averages of three measurements for each of the measurement values are calculated. Unlike in the method shown in FIG. 4, measurements are made in parallel and averages are formed.

After formation of the averages, in a difference formation loop (see FIG. 9b) the differences between the averaged measurements made on the left and the right arm are calculated. If the difference of the systolic average measurement is above 20 mmHg or if the difference between the diastolic average measurements is above 10 mmHg, a warning indicating a too high difference is displayed in the respective display 56 (see FIG. 6a). A result loop is then carried out (see FIG. 9c).

In a first step, the difference of the left and right systolic blood pressure values are determined. The difference (as an absolute value) is stored in a variable Diff_SBP. If the difference between the measurements is below 10 mm HG, by default, the left arm is used as the default measurement site. Subsequently, a “left arm” icon is displayed on the preferred measurement site display are 59 (see FIG. 6a).

If the difference between the systolic measurements on the right and the left arm is above 10 mm HG, it is first determined which measurement site leads to a higher measurement. If the systolic blood pressure measured at the left arm is higher, the left arm will be used and a left arm icon is displayed. If the right arm measurement is higher, the right arm will be used for subsequent measurements and a right arm icon is displayed.

The averaged blood pressure measurements of both measurement sites, i.e. of the right and the left arm are finally displayed.

These criteria of selection of a preferred measurement arm basically correspond to the ESH 2003 guidelines. The averaged results on the basis of the measurement on each of the arms may be used for verifying purposes.

FIG. 10 shows a flow chart of operation in the follow up mode. After having the arms supported at the appropriate height, the patient is asked to rest for 5 minutes. Measurement is then started. The cuff preferably is initially applied on the preferred arm. It can also be applied after the rest. On the decided arm, an ordinary automatic blood pressure measurement is made and the results are saved. In particular, systolic, diastolic blood pressures but also pulse rate, pulse pressure and mean arterial pressure may be determined and stored in a manner known to those skilled in the art.

After a rest of 60 seconds, a second blood pressure measurement is made on the same arm. The results of the second measurement are stored.

In a further step, the absolute values of the differences between the systolic and the diastolic blood pressure values of the first and second measurement are determined and stored in a variable Sd and Dd, respectively.

In a next step, it is determined whether the difference between the first and second measurements is above 5 mm HG.

If the difference between the systolic and diastolic blood pressure values measured in the first and second measurement is below 5 mm HG, an average systolic and diastolic blood pressure is formed and displayed and saved in a final step.

If the difference between the measurement is above 5 mm HG, the patient is asked to rest for another 60 seconds and a third blood pressure measurement is carried out. The results are saved. In a final step, an average of the three systolic and diastolic blood pressure measurement results is formed and displayed as an average and subsequently saved.

1-27. (canceled)

28. A system for blood pressure measurement, in particular for measurement at a point of care, the system comprising
a blood pressure measuring device for measuring the blood pressure of an individual
a seat for said individual adapted for properly seating said individual for correct blood pressure measurements,
wherein said seat includes means for ensuring correct position of the individual's feet.

29. A system for blood pressure measurement, the system comprising
a blood pressure measuring device for measuring the blood pressure of a patient, said blood pressure measuring device having a communication interface for transmission of measured blood pressure data,
a data collection device having a communication interface for receiving data transmitted by said blood pressure measuring device,
wherein said data collection device is arranged at a location physically separated from a location of said blood pressure measuring device in such a way that a direct contact between a doctor or a care person and the individual during blood pressure measurement can be prevented.

30. A system according to claim 29, wherein said blood pressure measuring device is arranged at a separated location at the point of care, said location being adapted for blood pressure measurements according to predetermined measurement criteria.

31. A system for blood pressure measurement, the system comprising
a blood pressure measuring unit
a first cuff for blood pressure measurement, adapted to be placed around a patient's left arm and connected to or connectable to said unit and
a second cuff for blood pressure measurement, adapted to be placed around a patient's right arm and connected to or connectable to said unit,
wherein the blood pressure measurement unit is provided with means for parallel measurement of the blood pressure with said first and said second cuff, in particular for simultaneous measurement.

32. A system according to claim 31, said system being operatable in at least two operating modes,
wherein one of said operating modes is an initial visit mode and
wherein in an initial visit loop carried out in said initial visit mode, a parallel measurement of the blood pressure with the first and the second cuff is made.

33. A system according to claim 31, wherein the system comprises means for determining a difference of blood pressure values measured on the right arm and blood pressure values measured on the left arm of the individual (I).

34. A system according to claim 32, wherein in addition to the initial visit mode, the system is operatable in at least one of a manual mode and a follow up mode,
wherein in the manual mode, current pressure values in at least one of said cuffs are instantly displayed on an instant pressure display area of said unit and
wherein in said follow up mode, blood pressure measurements are automatically made on the basis of the pressure signal provided by only one of said first and second cuff.

35. A system according to claim 34, wherein said system is provided with switching means for switching between at least two modes selected from the group of an initial visit mode, a follow up mode and a manual mode.

36. A system according to claim 31, wherein the system comprises means for determining a preferred measurement arm for subsequent blood pressure measurements and wherein the system preferably comprises a display means for indication of the preferred measurement arm, wherein preferably three measurements are subsequently taken and averages are used for determination of the preferred measurement arm for subsequent blood pressure measurement.

37. A system according to claim 31, wherein said system comprises means for forming average of measurement values of subsequent measurement.

38. A system according to claim 37, wherein the system comprises means for making a pause of a predetermined or predeterminable length between subsequent measurements.

39. A system according to claim 34, wherein said system comprises an input interface for manually entering blood pressure data during said initial visit loop.

40. A system according to claim 28, wherein said seat includes at least one of an arm support and a back support for said individual.

41. A system according to claim 29, wherein said data collection device further includes a communication interface for transmitting blood pressure data to a central data base.

42. A system according to claim 29, wherein said system includes means for generating reports in accordance with predetermined reporting criteria, particular in accordance with pay for performance criteria.

43. A system according to claim 33, wherein the system comprises means for prediction of arterial diseases based on interarm differences, in particular if the interarm differences of the systolic blood pressure are above 20 mm HG and/or the interarm difference of the diastolic blood pressure are above 10 mm HG.

44. A system according to claim 31, the system comprising a first deflation valve for deflation of the first cuff and a second deflation valve for deflation of the second cuff
wherein the deflation rate of the deflation valves is independently adjustable.

45. A method for blood pressure measurement of an individual at a point of care, comprising the steps of
placing said individual at a location in such a way that there is no direct contact between the individual and a doctor or care person during blood pressure measurement
automatically carrying out a blood pressure measurement of said individual with a blood pressure measuring device in absence of a doctor or a care person for gathering blood pressure data
transmitting said blood pressure data to a data collection device arranged at a location physically separated from said blood pressure measuring device.

46. A method according to claim 45, wherein prior to the measurement of blood pressure data, at least one instruction selected from the group of instructions to have feet properly positioned, to have arms supported, to place feet on a predetermined foot support and to take a rest for a predetermined period of time are given to the individual.

47. A method for blood pressure measurement of an individual at a point of care, wherein prior to blood pressure measurement, in a judgement step a judgement is made as to whether or not this is the individual's initial visit.

48. A method according to claim 47, wherein in the initial visit loop manually measured blood pressure data are entered into the system prior to automatic measurement.

49. A method according to claim 47,
wherein in the initial visit loop, measurement of the blood pressure of the individual are carried out on the individual's left and right arm in order to determine blood pressure values of the right arm and blood pressure values of the left arm and wherein preferably differences of the blood pressure values of the right arm and of the blood pressure values of the left arm are formed.

50. A method according to claim 49, wherein measurements are simultaneously made on the right and the left arm.

51. A method according to claim 47, wherein at least two subsequent measurements are taken and wherein an average of blood pressure values of subsequent measurements is determined.

52. A method according to claim 51, wherein a pause of a predetermined or predeterminable length is made between subsequent measurements.

53. A method according claim 51, wherein a difference between blood pressure values of two subsequent measurements is formed and wherein a further measurement is carried out if such difference is above a predetermined or predeterminable value.

54. A method for determining the blood pressure of an individual, wherein measurement of the blood pressure of the individual is carried out on the individual's left and right arm simultaneously with a first cuff placed around a patient's left arm and with a second cuff for blood pressure measurement placed around the patient's right arm,
whereby in a first step, the first and the second cuff each are inflated to a preset pressure and
whereby in a second step, the pressure within the cuffs is individually reduced by activation of a deflation valve associated with each of the cuffs,
wherein the deflation rate of each of the deflation valves is set in such a way that the total time for inflation and deflation for both cuffs is the same.
